(19) United States
(12) Patent Application Publication
(10) Pub. No.: US 2021/0154377 A1
(43) Pub. Date: May 27, 2021

(54) CONTROL SYSTEMS FOR MANAGEMENT OF FLUID BUILDUP INCLUDING PLEURAL EFFUSION AND ASCITES

(71) Applicant: KCI Licensing, Inc., San Antonio, TX (US)

(72) Inventors: Benjamin Andrew PRATT, Poole (GB); Christopher Brian LOCKE, Bournemouth (GB)

(21) Appl. No.: 17/254,654

(22) PCT Filed: Mar. 11, 2019
(86) PCT No.: PCT/US2019/021547

§ 371 Date: Dec. 21, 2020

Related U.S. Application Data
(60) Provisional application No. 62/691,086, filed on Jun. 28, 2018.

(51) Int. Cl. A61M 1/00

(52) U.S. Cl. CPC A61M 1/0033; A61M 1/0027; A61M 2202/0401; A61M 2202/0492; A61M 2205/50; A61M 2210/101; A61M 2210/1017; A61M 2205/18; A61M 2205/3389

(57) ABSTRACT

An apparatus for managing fluid buildup in an internal cavity that may allow automated or semi-automated management of fluid at the treatment site. The apparatus may include a drain fluidly coupled to a negative-pressure source and a vent valve. The may further include a control system configured to operate the negative-pressure source to manage fluid buildup at the treatment site. The apparatus may be particularly advantageous for management of fluid buildup in serous cavities, such as pleural effusion or ascites.

### RELATED APPLICATION

This application claims the benefit, under 35 U.S.C § 119(e), of the filing of U.S. Provisional Patent Application Ser. No. 62/691,086, entitled “CONTROL SYSTEMS FOR MANAGEMENT OF FLUID BUILDUP INCLUDING PLEURAL EFFUSION AND ASCITES,” filed Jun. 28, 2018, which is incorporated herein by reference for all purposes.

### TECHNICAL FIELD

The invention set forth in the appended claims relates generally to fluid management systems and more particularly, but without limitation, to control systems for management of fluid buildup including pleural effusion and ascites.

### BACKGROUND

Effusion refers to an abnormal accumulation of fluid in a body cavity. The most common sites of effusion include serous cavities, such as the pleural cavity (pleural effusion) and the peritoneal cavity (ascites). Effusion can produce considerable amounts of liquid, such as transudates and exudates. If not properly addressed, the accumulation of liquid can lead to infection, compression of internal structures, reduction of blood supply to the area, and even tissue death.

Regardless of the etiology of effusion, whether infection, trauma, medications, chemotherapy, or another cause, proper care of effusion is important to the outcome. Medical drainage devices are often used in treating effusion to address the production of fluids. However, common drainage devices, systems, and methods often face challenges with managing the drainage of fluids from within a body cavity.

While the clinical benefits of managing the accumulation of fluid in a body cavity are known, improvements to drainage devices, components, and processes may benefit healthcare providers and patients.

### BRIEF SUMMARY

New and useful systems, apparatuses, and methods for fluid management are set forth in the appended claims. Illustrative embodiments are also provided to enable a person skilled in the art to make and use the claimed subject matter.

For example, in some embodiments, an apparatus for managing fluid buildup at a treatment site includes a drain fluidly coupled to a negative-pressure source and a vent valve. The apparatus further includes a control system configured to operate the negative-pressure source to manage fluid buildup at the treatment site. In some examples, the apparatus may be configured to determine a nominal volume of the system prior to removing fluid, and may determine if there is enough fluid in a treatment site to remove. The apparatus may apply negative pressure to remove fluid to a container until a reduced pressure in the treatment site is detected. After removal, the apparatus may assess the change in system volume, which can allow the apparatus to monitor and estimate a fluid level in the container. Feedback may be given to an operator prior to a fluid-removal cycle that may result in a full canister.

In some examples, determining a nominal system volume may include running an automated volume calculation sequence based on dead-space detection algorithms, which can allow the system to have a benchmark for subsequent comparisons. An example dead-space detection algorithm may include closing a valve to isolate the system from the ambient environment, and operating the negative-pressure source to take the system to a preset pressure. Once a target pressure is reached, the valve may be opened to allow fluid flow at a known rate and to allow pressure in the treatment site to rise. A time to reach a pre-defined pressure can be measured and used as a basis for the volume calculation. The algorithm may be repeated and an average may be taken to account for movement of a patient.

Determining if there is enough fluid to remove can include running a control algorithm to check for fluid in the system at a set interval. In some examples, the set interval may be dynamic to reduce patient impact and maximize battery life (based upon monitoring how often fluid is removed). An example of a suitable algorithm may include opening a vent valve and operating a negative-pressure source to reduce pressure at a treatment site. Pressure data from two sensors may be monitored for a sample interval, and compared to a divergence threshold. If a difference between pressure data from the two sensors exceeds the divergence threshold within the sample interval, the fluid exceeds a fluid-removal threshold.

More generally, some illustrative embodiments of an apparatus for managing fluid in an internal cavity may comprise a drain fluidly coupled between a negative-pressure source and a vent valve. The drain may provide fluid communication with the treatment site and allow for the removal of fluids from the treatment site. A container and a first sensor may be fluidly coupled between the drain and the negative-pressure source. Additionally, a second sensor may be in fluid connection with the drain and a vent valve. The apparatus may further comprise a controller coupled to the negative-pressure source, the first sensor, the second sensor, and the vent valve. The sensors may measure operating parameters and provide feedback signals to the controller. The controller can be configured to operate one or more components of the apparatus to remove fluid from the treatment site. In some embodiments, the controller may be configured to determine an initial system volume, determine if fluid in the internal cavity exceeds a fluid-removal threshold, and operate the negative-pressure source to perform a fluid-removal cycle if the fluid exceeds the fluid-removal threshold.

Some example embodiments of an apparatus for managing fluid may comprise a negative-pressure source configured to be fluidly coupled to a first conduit, a first pressure sensor fluidly coupled to the negative-pressure source, a second pressure sensor configured to be fluidly coupled to a second conduit, a vent valve configured to be fluidly coupled to the second conduit, and a controller coupled to the negative-pressure source, the first pressure sensor, the second pressure sensor, and the vent valve. The controller can be configured to determine an initial system volume, operate the negative-pressure source to reduce pressure, open the vent valve, receive pressure data from the first pressure sensor and the second pressure sensor for a sample interval, determine if a difference between pressure data from the first pressure sensor and the second pressure sensor exceeds a divergence threshold within the sample interval, and if the difference exceeds the divergence threshold within the sample interval: open the vent valve, operate the negative-pressure source to reduce pressure, receive pressure data from the second pressure sensor, and deactivate the negative-pressure source if the pressure data indicates an increase in negative pressure.

The apparatus may be beneficial for various modes of treatment and for various types of treatment sites, and may be particularly advantageous for management of fluid buildup in serous cavities, such as pleural effusion or ascites. Other objectives, advantages, and a preferred mode of making and using the claimed subject matter may be understood best by reference to the accompanying drawings in conjunction with the following detailed description of illustrative embodiments.

### BRIEF DESCRIPTION OF THE DRAWINGS

FIG. 1 is a functional block diagram of an example embodiment of a fluid management system that can provide treatment in accordance with this specification.

FIG. 2 is a schematic diagram illustrating additional details that may be associated with some example embodiments of the fluid management system of FIG. 1.

FIG. 3 is a schematic diagram of an example of the fluid management system of FIG. 1 applied to an example cavity.

FIG. 4 is a flow chart illustrating a method of managing fluid that may be associated with some embodiments of the fluid management system of FIG. 1.

### DESCRIPTION OF EXAMPLE EMBODIMENTS

The following description of example embodiments provides information that enables a person skilled in the art to make and use the subject matter set forth in the appended claims, but it may omit certain details already well-known in the art. The following detailed description is, therefore, to be taken as illustrative and not limiting.

The example embodiments may also be described herein with reference to spatial relationships between various elements or to the spatial orientation of various elements depicted in the attached drawings. In general, such relationships or orientation assume a frame of reference consistent with or relative to a patient in a position to receive treatment. However, as should be recognized by those skilled in the art, this frame of reference is merely a descriptive expedient rather than a strict prescription.

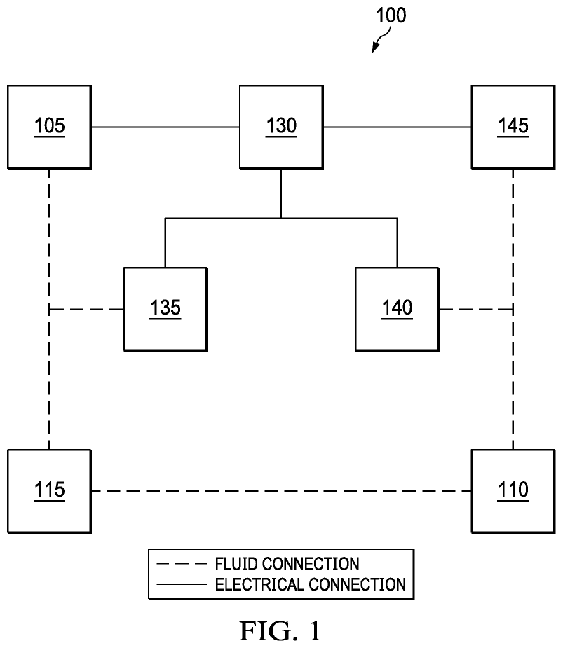

FIG. 1 is a simplified functional block diagram of an example embodiment of a fluid management system 100 that can provide negative-pressure to a treatment site in accordance with this specification.

The term “treatment site” in this context broadly refers to a body cavity, including, but not limited to, serous cavities. Serous cavities may include a pleural cavity or peritoneal cavity, for example.

The fluid management system 100 may include a source or supply of negative pressure, such as a negative-pressure source 105, and one or more distribution components. A distribution component is preferably detachable and may be disposable, reusable, or recyclable. A drain, such as a drain 110, and a fluid container, such as a container 115, are examples of distribution components that may be associated with some examples of the fluid management system 100.

A fluid conductor is another illustrative example of a distribution component. A “fluid conductor,” in this context, broadly includes a tube, pipe, hose, conduit, or other structure with one or more lumina or open pathways adapted to convey a fluid between two ends. Typically, a tube is an elongated, cylindrical structure with some flexibility, but the geometry and rigidity may vary. Moreover, some fluid conductors may be molded into or otherwise integrally combined with other components. Distribution components may also include or comprise interfaces or fluid ports to facilitate coupling and de-coupling other components.

The fluid management system 100 may also include a regulator or controller, such as a controller 130. Additionally, the fluid management system 100 may include sensors to measure operating parameters and provide feedback signals to the controller 130 indicative of the operating parameters. As illustrated in FIG. 1, for example, the fluid management system 100 may include a first sensor 135 and a second sensor 140 coupled to the controller 130.

The fluid management system 100 may also include a vent valve. For example, a vent valve 145 may be fluidly coupled to the drain 110 and electrically coupled to the controller 130, as illustrated in the example embodiment of FIG. 1.

Some components of the fluid management system 100 may be housed within or used in conjunction with other components, such as sensors, processing units, alarm indicators, memory, databases, software, display devices, or user interfaces that further facilitate fluid management. In some embodiments, the negative-pressure source 105 may be combined with the controller 130, the first sensor 135, the second sensor 140, the vent valve 145, and other components, into a fluid management treatment unit.

In general, components of the fluid management system 100 may be coupled directly or indirectly. For example, the negative-pressure source 105 may be directly coupled to the container 115 and may be indirectly coupled to the drain 110 through the container 115. Coupling may include fluid, mechanical, thermal, electrical, or chemical coupling (such as a chemical bond), or some combination of coupling in some contexts. For example, the negative-pressure source 105 may be electrically coupled to the controller 130 and may be fluidly coupled to one or more distribution components to provide a fluid path to a treatment site. In some embodiments, components may also be coupled by virtue of physical proximity, being integral to a single structure, or being formed from the same piece of material.

A negative-pressure supply, such as the negative-pressure source 105, may be a reservoir of air at a negative pressure or may be a manual or electrically-powered device, such as a vacuum pump, a suction pump, a wall suction port available at many healthcare facilities, or a micro-pump, for example. “Negative pressure” generally refers to a pressure less than a local ambient pressure, such as the ambient pressure in a local environment external to a sealed therapeutic environment. Alternatively, the pressure may be less than a hydrostatic pressure associated at the treatment site. Unless otherwise indicated, values of pressure stated herein are gauge pressures. References to increases in negative pressure typically refer to a decrease in absolute pressure, while decreases in negative pressure typically refer to an increase in absolute pressure. While the amount and nature of negative pressure provided by the negative-pressure source 105 may vary according to treatment requirements, the pressure is generally a low vacuum, also commonly referred to as a rough vacuum, between −5 mm Hg (−667 Pa) and −500 mm Hg (−66.7 kPa). In some examples, the negative-pressure source 105 may be configured to provide a negative pressure in a range of about 50 mm Hg to about 75 mm Hg for drainage.

The fluid mechanics of using a negative-pressure source to reduce pressure in another component or location, such as at a treatment site, can be mathematically complex. However, the basic principles of fluid mechanics applicable to reducing pressure at a treatment site are generally well-known to those skilled in the art, and the process of reducing pressure may be described illustratively herein as “delivering,” “distributing,” or “generating” negative pressure, for example.

In general, exudate and other fluid flow toward lower pressure along a fluid path. Thus, the term “downstream” typically implies something in a fluid path relatively closer to a source of negative pressure or further away from a source of positive pressure. Conversely, the term “upstream” implies something relatively further away from a source of negative pressure or closer to a source of positive pressure. Similarly, it may be convenient to describe certain features in terms of fluid “inlet” or “outlet” in such a frame of reference. This orientation is generally presumed for purposes of describing various features and components herein. However, the fluid path may also be reversed in some applications, such as by substituting a positive-pressure source for a negative-pressure source, and this descriptive convention should not be construed as a limiting convention.

The drain 110 can be generally adapted for insertion into treatment sites, such as body cavities. The drain 110 may take many forms, and have many sizes, shapes, or thicknesses, depending on a variety of factors, such as the type of treatment site or the nature and size of the treatment site. For example, the size and shape of the drain 110 may be adapted to the contours of a pleural cavity.

In some embodiments, the drain 110 may comprise or consist essentially of a fluid conductor. A fluid conductor in this context may comprise a means for transmitting negative pressure or collecting fluid from a treatment site under negative pressure. For example, the drain 110 may be adapted to receive negative-pressure from a source and distribute negative-pressure through a fluid conductor across the drain 110, which may have the effect of collecting fluid from a treatment site and drawing the fluid toward the source.

The container 115 is representative of a container, canister, pouch, or other storage component, which can be used to manage exudates and other fluids withdrawn from a treatment site. In many environments, a rigid container may be preferred or required for collecting, storing, and disposing of fluids. In other environments, fluids may be properly disposed of without rigid container storage, and a re-usable container could reduce waste and costs associated with negative-pressure therapy.

A controller, such as the controller 130, may be a microprocessor or computer programmed to operate one or more components of the fluid management system 100, such as the negative-pressure source 105. In some embodiments, for example, the controller 130 may be a microcontroller, which generally comprises an integrated circuit containing a processor core and a memory programmed to directly or indirectly control one or more operating parameters of the fluid management system 100. Operating parameters may include the power applied to the negative-pressure source 105, the pressure generated by the negative-pressure source 105, or the pressure distributed to the drain 110, for example. The controller 130 may also be configured to receive one or more input signals, such as a feedback signal, and programmed to modify one or more operating parameters based on the input signals.

Sensors, such as the first sensor 135 and the second sensor 140, are generally known in the art as any apparatus operable to detect or measure a physical phenomenon or property, and generally provide a signal indicative of the phenomenon or property that is detected or measured. For example, the first sensor 135 and the second sensor 140 may be configured to measure one or more operating parameters of the fluid management system 100. In some embodiments, the first sensor 135 may be a transducer configured to measure pressure in a pneumatic pathway and convert the measurement to a signal indicative of the pressure measured. In some embodiments, for example, the first sensor 135 may be a piezo-resistive strain gauge. The second sensor 140 may optionally measure operating parameters of the negative-pressure source 105, such as a voltage or current, in some embodiments. Preferably, the signals from the first sensor 135 and the second sensor 140 are suitable as an input signal to the controller 130, but some signal conditioning may be appropriate in some embodiments. For example, the signal may need to be filtered or amplified before it can be processed by the controller 130. Typically, the signal is an electrical signal, but may be represented in other forms, such as an optical signal.

In some embodiments, the controller 130 may receive and process data from one or more sensors, such as the first sensor 135. The controller 130 may also control the operation of one or more components of the fluid management system 100 to manage the pressure delivered to the drain 110. In some embodiments, the controller 130 may include an input for receiving a desired target pressure and may be programmed for processing data relating to the setting and inputting of the target pressure to be applied to the drain 110. In some example embodiments, the target pressure may be a fixed pressure value set by an operator as the target negative pressure desired for a treatment site and then provided as input to the controller 130. The target pressure may vary based on the type of treatment site, the type of injury or wound (if any), the medical condition of the patient, and the preference of an attending physician. After selecting a desired target pressure, the controller 130 may operate the negative-pressure source 105 in one or more control modes based on the target pressure and may receive feedback from one or more sensors to continue reducing pressure at the drain 110, maintain the target pressure at the drain 110, or operate the vent valve 145.

In some embodiments, the vent valve 145 may be opened or closed by the controller 130 to allow pressure at the treatment site to be normalized prior to or after performing measurements or functions. For example, the vent valve 145 may allow any exerted pressure due to movement of the patient to be normalized prior to performing a dead-space detection. In operation, the vent valve 145 may be opened after fluid removal to allow pressure at the treatment site to return to atmospheric pressure.

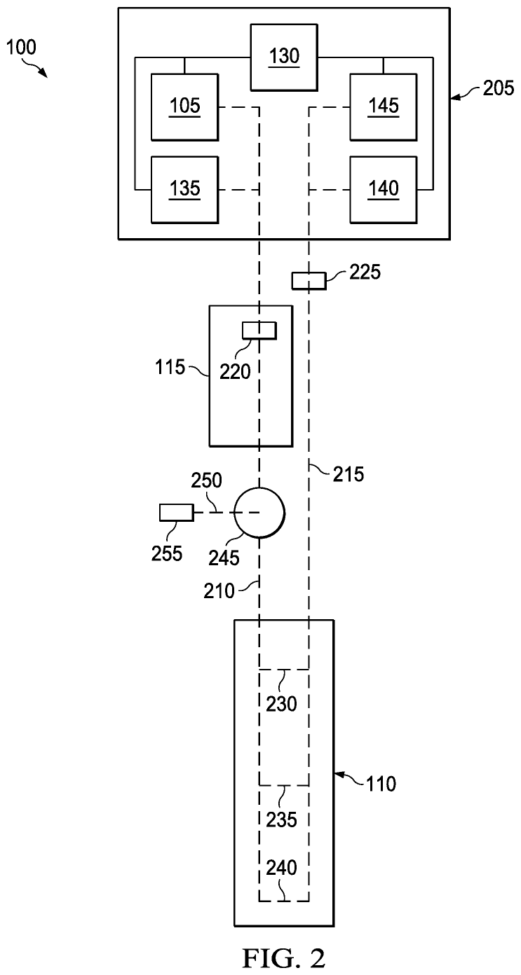

FIG. 2 is a schematic diagram illustrating additional details that may be associated with some example embodiments of the fluid management system 100. In the example embodiment of FIG. 2, the fluid management system 100 generally includes a source or supply of negative-pressure, such as the negative-pressure source 105, and one or more distribution components, such as the drain 110 and the container 115. The fluid management system 100 may also include a regulator or controller, such as the controller 130. Additionally, the fluid management system 100 may include sensors electrically coupled to the controller 130 to measure operating parameters and provide feedback signals to the controller 130 indicative of the operating parameters, such as the first sensor 135 and the second sensor 140. In the example of FIG. 2, the negative-pressure source 105 is combined with the controller 130, the first sensor 135, the second sensor 140, the vent valve 145, and other components into a fluid management treatment unit 205.

In some illustrative embodiments of the fluid management system 100, the treatment unit 205 may be fluidly coupled to the container 115 and the drain 110 through a first conduit 210, as depicted in FIG. 2. Further, the treatment unit 205 may be fluidly coupled to the drain 110 through a second conduit 215.

One or more filters, such as a first filter 220 and a second filter 225, may be placed in a fluid path between the drain 110 and other components to prevent damage, contamination, or both. For example, the first filter 220 may be placed in the fluid path of the first conduit 210, between the drain 110 and the negative-pressure source 105, and the second filter 225 may be placed in the fluid path of the second conduit 215, between the drain and the valve 145. In some embodiments, the filters may be antimicrobial, hydrophobic, or both.

As illustrated in the example of FIG. 2, some embodiments of the drain 110 may comprise one or more cross-conductors between the first conduit 210 and the second conduit 215. In FIG. 2, for example, the cross-conductors comprise a proximal conduit 230, an intermediate conduit 235, and a distal conduit 240, each of which fluidly couples the first conduit 210 to the second conduit 215.

In some embodiments, the fluid management system 100 may also comprise one or more valves for isolating certain components. For example, in some embodiments of the fluid management system 100, an isolation valve 245 may be fluidly coupled to the first conduit 210 between the container 115 and the drain 110, as depicted in FIG. 2. Additionally, the isolation valve 245 may be fluidly coupled to a third conduit 250 and a vent 255. The vent 255 may be an orifice or vent valve configured to open at a fixed pressure, for example. In some illustrative embodiments, the controller 130 may be configured to operate the isolation valve 245 to selectively couple the container 115 to the drain 110 or isolate the container 115 from the drain 110.

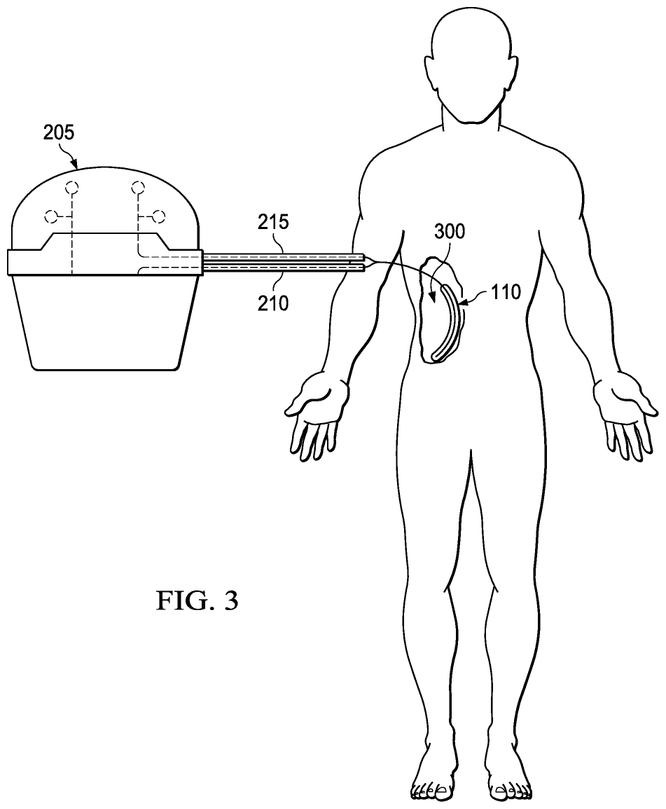

FIG. 3 is a schematic diagram of an example of the fluid management system 100, in which the drain 110 is inserted into a cavity 300. In the example of FIG. 3, the negative-pressure source 105 is in direct fluid connection to the cavity 300 via the conduit 210 and the container 115. The first sensor 135 in the treatment unit 205 can be fluidly connected to the container 115 and the negative-pressure source 105. The second sensor 140 is connected to the valve 145, the second filter 225, and the second conduit 215, which can allow the second sensor 140 to measure pressure along the full length of the drain 110 to the distal tip.

In general, when first applied, the controller 130 can run an automated volume calculation sequence based on one or more dead-space detection algorithms to determine a benchmark for subsequent comparison. To calculate a benchmark, in some embodiments the controller 130 may close the valve 145 and activate the negative-pressure source 105 to reduce pressure in the cavity 300 to a target pressure, which may be preconfigured or entered manually. Once target pressure is reached, the controller 130 can open the valve 145 to allow ambient air through the conduit 215, which can increase pressure in the cavity 300. The controller 130 can determine the time to reach a predetermined pressure. The time can be used as a basis for calculating the volume, or change in volume. In some examples, the process can be repeated and an average taken to compensate for movement of the patient or other variables. Periodically, the treatment unit 205 can determine if liquid in the cavity 300 exceeds a threshold and use the negative-pressure source 105 to remove the liquid from the cavity 300 if appropriate. The liquid can be stored in the container 115. In some examples, the controller 130 may optionally determine if there is sufficient capacity in the container 115 before removing the liquid from the cavity 300, and may alert an operator if there is insufficient capacity. In some embodiments, the controller 130 may be further programmed to store and process the initial system volume, the current system volume, and the volume change for each fluid-removal cycle performed. For example, such data can indicate changes in fluid rates, which can provide additional information about the state of the treatment or the patient.

In some embodiments, the fluid management system 100 may additionally or alternatively derive information from operating parameters such as pump duty and pressure rate of change. For example, if the negative-pressure source 105 is a pump set to a low duty but the pressure rate of change is increased, the current fluid capacity of the container 115 is reduced and the container 115 may be full. If the container 115 pressure reaches a target pressure in a pre-determined short duration, the container 115 may be full. This diagnostic may be automated such that a container-full condition may be determined and recorded with each cycle. The fluid management system 100 may be able to use this data to calculate the average fluid volume removed per fluid cycle over time and can be further used to report alarms if volumes depart from a historic trend.

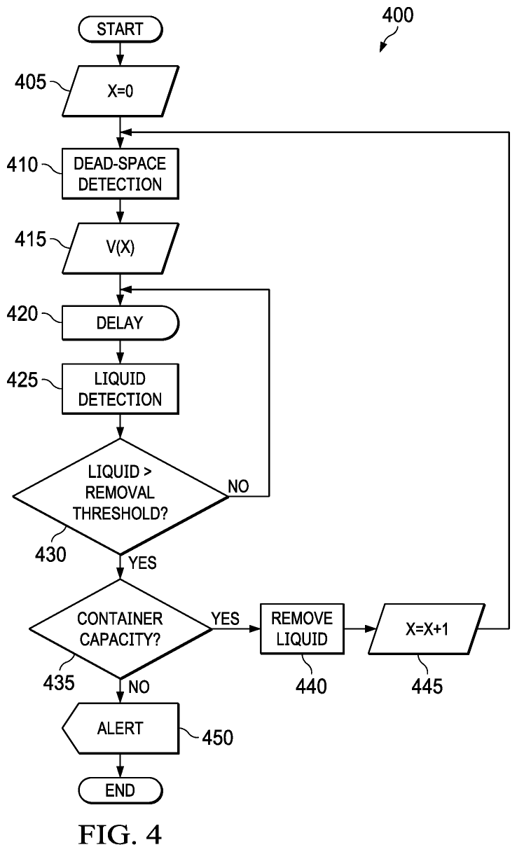

FIG. 4 is a flow chart illustrating a method 400 of managing fluid with a negative-pressure source. The method 400 may be associated with managing liquid in the cavity 300 of FIG. 3 with the fluid management system 100, for example. In some embodiments, the method may be implemented in a controller, such as the controller 130 in the fluid management system 100. The controller 130 may be configured to receive one or more input signals, such as pressure data from the first sensor 135 and the second sensor 140, and may be programmed to modify one or more operating parameters based on the input signals.

Referring to FIG. 4 for illustration, the method 400 may include initializing a counter variable. For example, a variable X may be set to 0 (X=0) at step 405. Dead-space detection can be used at step 410 to determine an initial system volume, which can be stored as V(0) at step 415. For example, in some embodiments, dead-space detection at step 410 may include closing the vent valve 145 and supplying a negative-pressure to the drain 110 through the conduit 210. Step 410 may further include deactivating the negative-pressure source 105 and opening the vent valve 145 to a pre-determined flow rate when pressure measured by the first sensor 135 is equal to a first target pressure. In some examples, a first target pressure of −125 mm Hg may be suitable. Opening the vent valve 145 may allow air to pass through the vent valve 145 into the cavity at the pre-determined flow rate, allowing pressure in the cavity to rise. Step 410 may further include monitoring pressure data from the first sensor 135 and determining a time for the pressure in the cavity to decay to a second target pressure. In some examples, the second target pressure may be atmospheric pressure. The decay time may be used as the basis for calculating the initial system volume. For example, the controller 130 may access a lookup table to correlate the decay time with a volume.

Liquid in the cavity can be checked periodically and removed as needed. The period may be a fixed interval in some embodiments, or may be adjusted dynamically. For example, the interval may be adjusted to reduce patient impact, maximize batter, or optimize other parameters. In some embodiments the controller 130 may be programmed to determine a set interval based upon how often liquid is determined to exceed a fluid-removal threshold. In the example of FIG. 4, the interval is represented as a delay at step 420.

After a delay at step 420, liquid in the cavity can be detected at step 425 and compared to a removal threshold at 430. For example, in some embodiments, step 425 may include supplying a negative-pressure to the first conduit 210 and monitoring pressure data received from both sides of the drain 110, which can be received from the first sensor 135 and the second sensor 140. Liquid in the cavity can cause a divergence between pressure measured by the first sensor 135 and the second sensor 140. The rate of divergence may be proportional to the amount of liquid in the cavity. If the pressure data at the first pressure sensor 135 and the second pressure sensor 140 diverge sufficiently within a sample interval, it may be determined that the liquid in the cavity exceeds the fluid-removal threshold.

If the liquid in the cavity exceeds the removal threshold at step 430, the capacity of the container 115 can be optionally evaluated at step 435. For example, in some embodiments, a volume of liquid in the cavity may be estimated by comparing the current dead-space space volume V(X) to the previous dead-space volume V(X−1). Additionally or alternatively, an average volume removed per cycle can be calculated and used to estimate the volume of liquid in the cavity. In some embodiments, the average fluid removed per cycle may be set to the upper limit of previous fluid-removal cycles to ensure that there is an acceptable factor of safety in the system. The volume of liquid in the cavity may be stored or saved in some examples. In some examples, changes in the volume over several cycles can be analyzed to determine changes in fluid rate, which can be indicative of treatment progress, complications, or other patient states.

Additionally or alternatively, determining the current fluid capacity of the container at step 435 may include a dead-space detection. For example, the isolation valve 245 may be operated to isolate the container 115 from the drain 110 and fluidly couple the container 115 to the third conduit 250, and determining capacity of the container based on dead-space detection similar or analogous to the dead-space detection of step 410. More specifically, in some examples, step 435 may further include operating the negative-pressure source 105 to reduce pressure in the container 115 until the pressure at the first sensor 135 is equal to a first target pressure. The first target pressure may be about −125 mm Hg, for example. Step 435 may additionally include deactivating the negative-pressure source 105. In some embodiments, the vent 255 may be configured to open at the first target pressure, or may be opened by the controller 130 at the first target pressure. Step 435 may further include monitoring pressure data from the first sensor 125 and determining a time to reach a second target pressure. The second target pressure may be atmospheric pressure in some embodiments. The time to reach the second target pressure may be used as the basis for calculating the current fluid capacity of the container 115. For example, the controller 130 may access a lookup table to correlate the decay time with a volume.

If the container 115 has capacity to receive the liquid from the cavity, the liquid can be removed at step 440. For example, removing the liquid at step 440 may include operating the negative-pressure source 105 to reduce pressure at the drain 110, which can draw fluid into the container 115. The vent valve 145 may also be opened to provide a set flow rate. Supplying negative pressure to the drain 110 may draw fluid and exudates into container 115. Step 440 may further include receiving pressure data from the second pressure sensor 140. Liquid in a cavity may block the drain 110, preventing negative pressure supplied by the negative-pressure source 105 from being transmitted to the second pressure sensor 140. Thus, the pressure measured at the second pressure sensor 140 may not indicate an increase in negative pressure until the liquid is removed from the cavity. Step 440 may include operating the negative-pressure source 105 to continue supplying negative-pressure to the drain 110 until the pressure data at the second pressure sensor 140 indicates an increase in negative pressure. Step 440 may further include deactivating the negative-pressure source 105 and opening the vent valve 145 to atmosphere when an increase in negative pressure is detected at the second pressure sensor 140. For example, opening the vent valve 145 can allow pressure at the treatment site to be normalized prior to or after performing measurements or functions. Opening the vent valve to atmosphere may ensure that pressure in a cavity returns to a nominal atmospheric pressure.

The counter variable X can be incremented at step 445.

If the container 115 is full or otherwise does not have capacity to receive the liquid from the cavity, the controller 130 can generate an alert at step 450 and may shut-down the treatment unit 205. In some embodiments, determining whether performing the fluid-removal cycle could trigger a canister full condition at step 435 may include determining whether the average fluid removed per fluid-removal cycle is greater than the fluid capacity of the container 115.

The systems, apparatuses, and methods described herein may provide significant advantages. For example, utilizing existing therapy devices or those with minor adjustments with the systems, apparatuses, and methods described, may allow for automated or semi-automated management of fluid in internal cavities, only requiring the operator to perform insertion of the drain, activation of the apparatus, and canister changes. Automated or semi-automated management of fluid in internal cavities may also allow fluid removal to be performed at a dynamic interval, based upon learning how often fluid is removed, that may maximize battery life and reduce patient impact by managing pressure in the internal cavity to maximize patient comfort. The systems, apparatuses, and methods described herein may provide a simple and cost effective system for managing excess fluids which can reduce cost and improve the patient and operator's ability to manage fluid removal.

While shown in a few illustrative embodiments, a person having ordinary skill in the art will recognize that the systems, apparatuses, and methods described herein are susceptible to various changes and modifications that fall within the scope of the appended claims. Moreover, descriptions of various alternatives using terms such as “or” do not require mutual exclusivity unless clearly required by the context, and the indefinite articles “a” or “an” do not limit the subject to a single instance unless clearly required by the context. Components may be also be combined or eliminated in various configurations for purposes of sale, manufacture, assembly, or use. For example, in some configurations the dressing 110, the container 115, or both may be eliminated or separated from other components for manufacture or sale. In other example configurations, the controller 130 may also be manufactured, configured, assembled, or sold independently of other components.

The appended claims set forth novel and inventive aspects of the subject matter described above, but the claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described in the context of some embodiments may also be omitted, combined, or replaced by alternative features serving the same, equivalent, or similar purpose without departing from the scope of the invention defined by the appended claims.

1. An apparatus for managing fluid in an internal cavity, the apparatus comprising:
a drain;
a first conduit fluidly coupled to the drain;
a fluid container fluidly coupled to the first conduit;
a negative-pressure source fluidly coupled to the first conduit;
a first pressure sensor fluidly coupled to the first conduit between the fluid container and the negative-pressure source;
a second conduit fluidly coupled to the drain;
a second pressure sensor fluidly coupled to the second conduit;
a vent valve fluidly coupled to the second conduit; and
a controller coupled to the negative-pressure source, the first pressure sensor, the second pressure sensor, and the vent valve, the controller configured to:
perform a first dead-space detection to determine an initial system volume,
determine if fluid in the internal cavity exceeds a fluid-removal threshold, and
operate the negative-pressure source to perform a fluid-removal cycle if the fluid exceeds the fluid-removal threshold.

2. The apparatus of claim 1, wherein performing the first dead-space detection to determine the initial system volume comprises:
closing the vent valve;
operating the negative-pressure source to reduce pressure at the drain;
deactivating the negative-pressure source and opening the vent valve at a first time when pressure measured by the first pressure sensor is equal to a first negative pressure;
determining a second time when pressure measured by the first pressure sensor is equal to a second negative pressure; and
determining the initial system volume based on a difference between the first time and the second time.

3. The apparatus of claim 2, wherein the first negative pressure is about 125 mm Hg.

4. The apparatus of claim 2, wherein the second negative pressure is about 0 mm Hg.

5. The apparatus of claim 1, wherein determining if fluid in the internal cavity exceeds the fluid-removal threshold comprises:
operating the negative-pressure source to reduce pressure;
opening the vent valve;
receiving pressure data from the first pressure sensor and the second pressure sensor for a sample interval; and
determining that the fluid in the internal cavity exceeds the fluid-removal threshold if a difference between pressure data from the first pressure sensor and the second pressure sensor exceeds a divergence threshold within the sample interval.

6. The apparatus of claim 1, wherein the fluid-removal cycle comprises:
opening the vent valve;
operating the negative-pressure source to reduce pressure in the first conduit;
receiving pressure data from the second pressure sensor;
deactivating the negative-pressure source if the pressure data indicates an increase in negative pressure; and
opening the vent valve to atmosphere.

7. The apparatus of claim 1, further comprising:
an isolation valve fluidly coupled to the first conduit between the drain and the fluid container;
a third conduit fluidly coupled to the isolation valve; and
a vent fluidly coupled to the third conduit;
wherein the isolation valve is configured to selectively couple the fluid container to the third conduit or the drain.

8. The apparatus of claim 7, wherein the controller is further configured to:
operate the isolation valve to fluidly isolate the fluid container from the drain;
operate the negative-pressure source to reduce pressure in the fluid container;
deactivate the negative-pressure source and open the vent at a first time when pressure measured by the first pressure sensor is equal to a first target pressure;
determine a second time when pressure measured by the first pressure sensor is equal to a second target pressure;
determine a fluid capacity of the fluid container based on a difference between the first time and the second time; and
operate the negative-pressure source to perform the fluid-removal cycle if the fluid capacity is sufficient to allow further removal of fluid.

9. The apparatus of claim 1, wherein the drain comprises at least one cross-conductor fluidly coupled to the first conduit and the second conduit.

10. The apparatus of claim 1, wherein the drain comprises:
a distal conduit fluidly coupled to the first conduit and the second conduit;
an intermediate conduit fluidly coupled to the first conduit and the second conduit; and
a proximal conduit fluidly coupled to the first conduit and the second conduit.

11. The apparatus of claim 1, wherein the controller is further configured to:
perform a second dead-space detection to determine a current system volume;
determine a volume change between the initial system volume and the current system volume;
determine a fluid capacity of the fluid container based on the volume change; and
determine that a fluid-removal cycle could be performed without triggering a canister full condition if the fluid capacity is greater than the volume change.

12. The apparatus of claim 11, wherein the controller is further configured to alert an operator if fluid volume exceeds the fluid capacity.

13. An apparatus for managing fluid in an internal cavity, the apparatus comprising:
a negative-pressure source configured to be fluidly coupled to a first conduit;
a first pressure sensor fluidly coupled to the negative-pressure source;
a second pressure sensor configured to be fluidly coupled to a second conduit;
a vent valve configured to be fluidly coupled to the second conduit; and
a controller coupled to the negative-pressure source, the first pressure sensor, the second pressure sensor, and the vent valve, the controller configured to:
determine an initial system volume,
operate the negative-pressure source to reduce pressure,
open the vent valve,
receive pressure data from the first pressure sensor and the second pressure sensor for a sample interval,
determine if a difference between pressure data from the first pressure sensor and the second pressure sensor exceeds a divergence threshold within the sample interval, and
if the difference exceeds the divergence threshold within the sample interval:
open the vent valve,
operate the negative-pressure source to reduce pressure,
receive pressure data from the second pressure sensor, and
deactivate the negative-pressure source if the pressure data indicates an increase in negative pressure.

14. (canceled)
